(19) United States
(12) Patent Application Publication
(10) Pub. No.: US 2006/0141055 A1
(43) Pub. Date: Jun. 29, 2006

(54) Cardiac development promoting activity of perivitelline fluid of embryos of Indian horseshoe crab, Tachypleus gigas (Muller)

(75) Inventors: Surendra Ghaskadbi, Pune (IN); Vidya Patwardhan, Pune (IN); Pradeep Bhaskar Parab, Pune (IN); Perunninakulath Subrayan Parameswaran, Dona Paula (IN); Anil Chatterji, Dona Paula (IN)

Correspondence Address: Ladas & Parry, 26 West 61 Street, New York, NY, 10023, US

(73) Assignee: COUNCIL OF SCIENTIFIC & INDUSTRIAL RESEARCH

(21) Appl. No.: 11/022,556

(22) Filed: Dec. 23, 2004

(51) Int. Cl. A61K 35/64

(52) U.S. Cl. 424/538

(57) ABSTRACT

The present invention relates to a method for cardiac development activity in a subject comprising administering to the subject an appropriate amount of perivitelline fluid obtained from fertilized eggs of horseshoe crab.

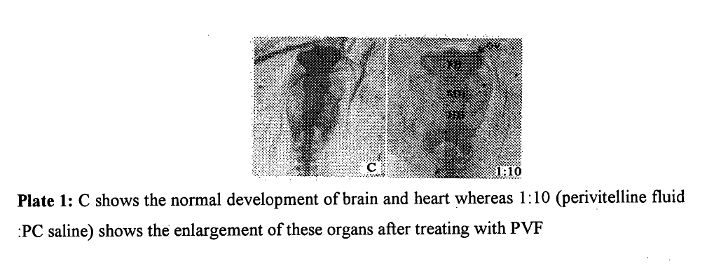

### FIELD OF INVENTION

The present invention relates to the use of perivitelline fluid of embryos of Indian horseshoe crab Tachypleus gigas (Müller) in cardiac development promoting activity. The present invention particularly relates to cardiac development promoting activity of perivitelline fluid of embryos of Indian horseshoe crab, Tachypleus gigas (Müller).

### BACKGROUND OF THE INVENTION

Organogenesis and cell replacement therapy is going to be one of the more popular methods to tackle several dreaded medical complications including diabetes and degenerative diseases related to heart. In vitro organ development and stem cell biology are two of the approaches that will underline the success of such a strategy. However, in order to achieve ideal organ growth, differentiation and/or regeneration, it will be necessary to identify, purify and employ specific molecules, either individually or in various combinations. The same is true for successful applications of stem cell biology where in, one needs to provide a defined milieu to naïve stem cell so as to coax them to take one or the other developmental pathway, at the will of the experimenter. The possible source for such novel molecules is marine animals that include the horseshoe crab and green mussels. It appears from the results of the present study that PVF of the Indian horseshoe crab contains peptide(s) capable of positively influencing differentiations of specific organs. Such peptides are likely to be present in minute quantities and may be as proteins in addition to the four major proteins reported in the Japanese horseshoe crab. (Nagai T, Kawabata S-I, Shishikura F, Sugita H: Purification, characterization, and amino acid sequence of an embryonic lectin in perivitelline fluid of the horseshoe crab. J. Biol. Chem. 274: 37673-37678, 1999).

Reference is made to a publication wherein purification, characterization and amino acid sequence of an embryonic lectin in perivitelline fluid of Japanese horseshoe crab has been reported. It is proposed that lectin TL-P may have an important role in completing embryonic development by interacting with endogenous glycoproteins or N-acetylhexosamines. (Nagai T, Kawabata S-I, Shishikura F, Sugita H: Purification, characterization, and amino acid sequence of an embryonic lectin in perivitelline fluid of the horseshoe crab. J. Biol. Chem. 274: 37673-37678, 1999).

Reference is made to another publication wherein evidence has been provided for the regulatory role of fibroblast growth factors 1 and 4 in chick cardiogenesis (Zhu K, Sasse J, McAllister D, Lough J: Evidence that fibroblast growth factors 1 and 4 participate in regulation of cardiogenesis. Developmental Dynamics, 207, pg.429-438, 1996).

Reference is made to another publication wherein evidence has been presented to show that activin or an activin-like molecule is required for and is sufficient to stimulate cardiac myogenesis in posterior region of chick pregastrula epiblast. (Yatskievich T. A, Ladd A N, Antin P B: Induction of cardiac myogenesis in avian pregastrula epiblast: the role of the hypoblast and activin. Development, 124, pg.2561-2570, 1997).

### OBJECTIVES OF THE INVENTION

The main objective of the present investigation is to identify and develop cardiac development promoting activity of perivitelline fluid of embryos of Indian horseshoe crab, Tachypleus gigas (Müller).

### SUMMARY OF THE INVENTION

Accordingly the present invention provides a method for promoting cardiac development activity in a subject comprising administering to the subject an appropriate amount of perivitelline fluid obtained from fertilized eggs of horseshoe crab.

In one embodiment of the invention, the subject is a mammal.

In another embodiment of the invention, the subject is a human being.

In yet another embodiment of the invention, the perivitelline fluid is diluted to a range of 1:10 to 1:100 prior to administration to the subject.

In yet another embodiment of the invention, the perivitelline fluid is collected from the fertilized eggs of the horseshoe crab aseptically, aliquoted and stored at −20° C.

### DETAILED DESCRIPTION OF THE INVENTION

The present invention relates to a method for promoting cardiac development activity in a subject by administering to the subject an appropriate amount of perivitelline fluid obtained from fertilized eggs of horseshoe crab. The subject is a mammal, such as a human being. The perivitelline fluid is diluted to a range of 1:10 to 1:100 prior to administration to the subject. The perivitelline fluid is collected from the fertilized eggs of the horseshoe crab aseptically, aliquoted and stored at −20° C.

The method has been validated according to the test results given below. The perivitelline fluid of embryos of Indian horseshoe crab, are collected aseptically from the fertilized eggs of the Indian horseshoe crab Tachypleus gigas, aliquoted and stored at −20° C. Chick embryos were cultured in vitro using New's single ring technique. Freshly laid White leghorn chicken eggs were incubated at 37.5° C. and humidity in the range of 90 to 95% for 16-18 hours to allow the embryos to reach HH stage 4. The shell was cleaned with 70% ethanol and broken on its broader end using a pair of blunt forceps after cooling the eggs to room temperature for 30 minutes. The thick albumin was then discarded and thin albumin was collected in a sterile beaker. The yolk ball was taken out in a sterile glass dish containing enough Pannet Compton saline (PC saline) to submerge the yolk ball completely and then cleaned of adhering thick albumin and chalazae. The vitelline membrane was cut along the equatorial level, keeping the blastoderm in the center and carefully peeled off along with the blastoderm. The vitelline membrane was transferred onto a watch glass, which was placed over a moistened cotton ring in a 90 mm diameter petri dish. The adhering yolk was carefully cleaned off as much as possible. A 20 mm diameter glass ring was placed on top of the vitelline membrane in such a way that the blastoderm was in the center with its ventral side up. The membrane was gently pulled and tucked over the ring to keep the blastoderm stretched and excess, if any, was trimmed. Thin albumin was placed in the watch glass under the ring as nourishment to the embryo. The glass ring was lifted so that the vitelline membrane bulged slightly inside the ring. The embryos were observed under dissection binocular and healthy and normal ones were staged using morphological criteria.

The cultures were randomly divided into two groups. The culture ring was completely removed by using a Pasteur pipette. Each embryo of the control group received 100 μl of PC saline while each embryo of the treatment group was treated with 100 μl of 1:10 diluted perivitelline fluid in PC saline. The embryos were left at room temperature for 30 minutes for diffusion of various molecules and then incubated at 37.5° C. for 22 hours. At the end of the incubation period embryos were examined to check the development of brain, heart and somites and detailed description was noted. The embryos were then detached from the vitelline membrane and fixed for whole mount preparations. Each embryo was fixed individually in glacial acetic acid and ethanol (1:3) on a glass slide. Care was taken to ensure that the embryos remained absolutely flat, and free of folds and wrinkles. The embryos were stored in the same fixative overnight and then transferred to 70% ethanol. The embryos were either stored in 70% ethanol at room temperature in tightly capped storage vials till use after several changes of 70% ethanol to remove all traces of acetic acid, or immediately hydrated in graded series of ethanol and finally distilled water.

After several washes in distilled water, embryos were stained with Delafield's hematoxylin for 15-20 minutes and de-stained in very dilute HCl till the structural details of neural tube and somites were clearly seen. The embryos were differentiated in dilute alkali water for 5 minutes and after a quick wash of distilled water for 5 minutes, embryos were dehydrated in graded series of ethanol. The embryos were counter stained with 0.5% eosin in 90% ethanol and after complete dehydration in 100% ethanol, embryos were placed in a mixture of ethanol and xylene (1:1) for 5 minutes, cleared in xylene for 3-5 minutes and mounted under a cover slip in DPX to make permanent preparations. Various morphological features of treated embryos were studied and compared to those of control embryos to understand of effects of treatment on morphogenesis of different embryonic organs.

The present invention relates to cardiac development promoting activity of perivitelline fluid of embryos of Indian horseshoe crab, Tachypleus gigas (Müller) and its probable role in cardiac development. The present investigation clearly demonstrate that the perivitelline fluid (PVF) from embryos of the Indian horseshoe crab has promoted embryonic growth in cultured chick embryos and also specifically stimulated the development and differentiation of brain and heart. Chick embryo explants cultured in vitro were used as a model system for our studies. This is a standard model for developmental studies on higher vertebrates since several aspects of chick embryo development are remarkably similar to mammalian development and are amenable to in vitro culture and experimentation.

Gastrulating chick embryos were allowed to develop in presence of various dilutions of PVF of horseshoe crab for several hours and the development and differentiation of specific organs and systems were monitored. The results showed that PVF tended to stimulate several aspects of chick embryonic development. The effects of the PVF were twofold. It stimulated the overall growth of the developing embryos as seen from the stimulation of axis elongation in treated embryos as compared to the controls. It also resulted in the stimulation of differentiation of specific organs, such as, brain and heart. It also interfered with the process of somitogenesis. All these effects clearly indicate that the PVF of the Indian horseshoe crab (T. gigas) contains molecules that stimulate growth, probably thorough increased mitosis as well as molecule(s) that influence differentiation of specific organs.

1) Collection of peri-vitelline Fluid From the Fertilized Eggs of the Horseshoe Crab:

Fertilized eggs of the horseshoe crab were collected from the nests located on the sandy beach at Balramgari (Orissa). The fertilized eggs were transferred in filtered seawater and incubated at a constant in artificial incubators. As soon as the eggs became transparent, showing the movement of trilobite larvae, the peri-vitelline fluid was collected aseptically, aliquot and stored at −20° C.

2) Culture of Chick Embryos:

Chick embryos were cultured in vitro using New's single ring technique. Freshly laid White leghorn chicken eggs were incubated at 37.5° C. and humidity in the range of 90 to 95% for 16-18 hours to allow the embryos to reach HH stage 4. After cooling the eggs to room temperature for 30 minutes, shell was cleaned with 70% ethanol and broken on its broader end using a pair of blunt forceps. Thick albumin was discarded and thin albumin was collected in a sterile beaker. The yolk ball was taken out in a sterile glass dish containing enough Pannet Compton saline (PC saline) to submerge the yolk ball completely. The yolk ball was cleaned of adhering thick albumin and chalazae. The vitelline membrane was cut along the equatorial level, keeping the blastoderm in the center and carefully peeled off along with the blastoderm. It was transferred onto a watch glass, which was placed over a moistened cotton ring in a 90-mm diameter petri dish. Adhering yolk was carefully cleaned off as much as possible. A glass ring with 20 mm diameter was placed on top of the vitelline membrane in such a way that the blastoderm was in the center with its ventral side up. Membrane was gently pulled and tucked over the ring to keep the blastoderm stretched and excess, if any, was trimmed. Thin albumin was placed in the watch glass under the ring as nourishment to the embryo. The glass ring was lifted so that the vitelline membrane bulged slightly inside the ring. Embryos were observed under dissection binocular and healthy and normal ones were staged using morphological criteria described by Hamburger and Hamilton (New 1966: In: The Culture of Vertebrate Embryos. Logos Press, London, pp. 47-84).

3) Treatment of Embryos:

HH stage 4 embryos were used to study the effects of perivitelline fluid. Cultures were randomly divided into two groups. Using a Pasteur pipette, PC saline remaining in the culture ring was completely removed. Each embryo of the control group received 100 μl of PC saline while each embryo of the treatment group was treated with 100 μl of 1:10 for experiment-I and 1:100 for experiment-2 of diluted perivitelline fluid in PC saline. Embryos were left at room temperature for 30 minutes for diffusion of various molecules and then incubated at 37.5° C. for 22 hours.

At the end of the incubation period embryos were examined to check the development of brain, heart and somites and detailed description was noted. The embryos were then detached from the vitelline membrane and fixed for whole mount preparations.

4) Preparation of Whole Mounts:

Each embryo was fixed individually in glacial acetic acid and ethanol (1:3) on a glass slide. Care was taken to ensure that the embryos remained absolutely flat, and free of folds and wrinkles. These were stored in the same fixative overnight and then transferred to 70% ethanol. After several changes of 70% ethanol to remove all traces of acetic acid, these embryos were either stored in 70% ethanol at room temperature in tightly capped storage vials till use or immediately hydrated in graded series of ethanol and finally distilled water. After several washes in distilled water, embryos were stained with Delafield's hematoxylin for 15-20 minutes and destained in very dilute HCl till the structural details of neural tube and somites were clearly seen. This was followed by differentiation in dilute alkali water for 5 minutes. After a quick wash of distilled water for 5 minutes, embryos were dehydrated in graded series of ethanol. Embryos were counter stained with 0.5% eosin in 90% ethanol. After complete dehydration in 100% ethanol, embryos were placed in a mixture of ethanol and xylene (1:1) for 5 minutes, cleared in xylene for 3-5 minutes and mounted under a cover slip in DPX to make permanent preparations.

Various morphological features of treated embryos were studied and compared to those of control embryos to understand of effects of treatment on morphogenesis of different embryonic organs (Tables 1 and 2; Plate 1).

The aforesaid process requires absolute precaution for bacterial and fungal contaminations at all above processing steps. All apparatus and reagents must therefore, be pyrogen free.

The novelty and inventive steps of the present invention is to identify a new cardiac development promoting activity in the peri-vitelline fluid collected from the fertilized eggs of the horseshoe crab.

The perivitelline fluid can be used for cardiac tissue regeneration by administration to a subject in vivo in therapeutic amounts.

The following illustrative examples of the method of the present invention should not be construed to limit the scope of the present invention.

### EXAMPLE-1

### Effects of Perivitelline Fluid (PVF) on Developing Chick Embryos

| | | Treated with 1:10 dil. |
|---|---|---|
| Feature | Control (n = 25) | PVF (n = 29) |
| Heart | Normal in 19 (76%) | Normal in 5 (17%) |
| | Small/undeveloped | Enlarged in 24 (83%) |
| | in 6 (24%) | |
| No. of | 9.4 ± 1.63 | 11.6 ± 1.3 |
| somites ± S.D. | | |

### EXAMPLE-2

### Effects of Perivitelline Fluid (PVF) on Developing Chick Embryos

| | | Treated with 1:100 dil. |
|---|---|---|
| Feature | Control (n = 25) | PVF (n = 28) |
| Heart | Normal in 20 (80%) | Normal in 7 (25%) |
| | Abnormal in 4 (16%) | Enlarged in 16 (57%) |
| | Underdeveloped | Underdeveloped in 5 (18%) |
| | in 1 (4%) | |
| No. of | 9 ± 1.4 | 10.46 ± 1.72 |
| somites ± S.D. | | |

Advantages of the Present Invention

1. Growth promoting effects of the PVF of Indian horseshoe crab has been identified.

2. The PVF predominantly promoted the development of heart. This property of the PVF can be exploited for use in vitro heart regeneration and cardiac stem cell biology.

### REFERENCES

1. Nagai T, Kawabata S-I, Shishikura F, Sugita H: Purification, characterization, and amino acid sequence of an embryonic lectin in perivitelline fluid of the horseshoe crab. J. Biol. Chem. 274: 37673-37678, 1999.
2. Zhu K, Sasse J, McAllister D, Lough J: Evidence that fibroblast growth factors 1 and 4 participate in regulation of cardiogenesis. Developmental Dynamics, 207, pg.429-438, 1996.
3. Yatskievich T A, Ladd A N, Antin P B: Induction of cardiac myogenesis in avian pregastrula epiblast: the role of the hypoblast and activin. Development, 124, pg.2561-2570, 1997.
4. New 1966: In: The Culture of Vertebrate Embryos. Logos Press, London, pp. 47-84.

We claim:

1. A method for cardiac development activity in a subject comprising administering to the subject an appropriate amount of perivitelline fluid obtained from fertilized eggs of horseshoe crab.

2. A method as claimed in claim 1 wherein the subject is a mammal.

3. A method as claimed in claim 1 wherein the subject is a human being.

4. A method as claimed in claim 1 wherein the perivitelline fluid is diluted to a range of 1:10 to 1:100 prior to administration to the subject.

5. A method as claimed in claim 1 wherein the perivitelline fluid is collected from the fertilized eggs of the horseshoe crab aseptically, aliquoted and stored at −20° C.
